(19) United States
(12) Patent Application Publication
(10) Pub. No.: US 2025/0144055 A1
(43) Pub. Date: May 8, 2025

(54) METHOD AND COMPOSITION FOR REDUCING MYOPIA PROGRESSION

(71) Applicant: THE HONG KONG POLYTECHNIC UNIVERSITY, Hong Kong (CN)

(72) Inventors: Chuen LAM, Hong Kong (CN); Jing Fang BIAN, Hong Kong (CN); Yan Yin TSE, Hong Kong (CN); Chi ho TO, Hong Kong (CN)

(21) Appl. No.: 18/501,136

(22) Filed: Nov. 3, 2023

(51) Int. Cl. A61K 31/19; A61P 27/10

(52) U.S. Cl. CPC A61K 31/19; A61P 27/10

(57) ABSTRACT

A method for reducing myopia progression. In particular, the method includes administering to a subject a compound represented by the following formula or a composition comprising an effective amount of the same:

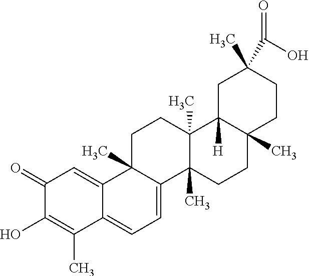

or a pharmaceutically accept salt thereof, in which the compound can target and inhibit catechol-O-methyltransferase, up-regulate retinal dopamine expression and change the choroidal thickness of a uvea of the subject, where an increase in choroidal thickness indicates a reduction in myopia progression.

### TECHNICAL FIELD

The present disclosure generally relates to a catechol-O-methyltransferase (COMT) inhibitor for reducing myopia progression, in particular, to celastrol or a pharmaceutically acceptable salt thereof for use in reducing myopia progression.

### BACKGROUND

The following references are cited and described herein:
1. FELDKAEMPER, M. & SCHAEFFEL, F. 2013. An updated view on the role of dopamine in myopia. Exp Eye Res, 114, 106-19.
2. ZHOU, X. T., PARDUE, M. T., IUVONE, P. M. & QU, J. 2017. Dopamine signaling and myopia development: What are the key challenges. Progress in Retinal and Eye Research, 61, 60-71.
3 STONE, R. A., LIN, T., LATIES, A. M. & IUVONE, P. M. 1989. Retinal dopamine and form-deprivation myopia. Proc Natl Acad Sci USA, 86, 704-6.4. SCHMID, K. L. & WILDSOET, C. F. 2004. Inhibitory effects of apomorphine and atropine and their combination on myopia in chicks. Optometry and Vision Science, 81, 137-147.
5. DONG, F., ZHI, Z. N., PAN, M. Z., XIE, R. Z., QIN, X. Y., LU, R. X., MAO, X. J., CHEN, J. F., WILLCOX, M. D. P., QU, J. & ZHOU, X. T. 2011. Inhibition of experimental myopia by a dopamine agonist: different effectiveness between form deprivation and hyperopic defocus in guinea pigs. Molecular Vision, 17, 2824-2834.
6. IUVONE, P. M., TIGGES, M., STONE, R. A., LAMBERT, S. & LATIES, A. M. 1991. Effects of Apomorphine, a Dopamine Receptor Agonist, on Ocular Refraction and Axial Elongation in a Primate Model of Myopia. Investigative Ophthalmology & Visual Science, 32, 1674-1677.
7. YAN, T. T., XIONG, W. W., HUANG, F. R., ZHENG, F., YING, H. F., CHEN, J. F., QU, J. & ZHOU, X. T. 2015. Daily Injection But Not Continuous Infusion of Apomorphine Inhibits Form-Deprivation Myopia in Mice. Investigative Ophthalmology & Visual Science, 56, 2475-2485.
8. FAUST, K., GEHRKE, S., YANG, Y. F., YANG, L. C., BEAL, M. F. & LU, B. W. 2009. Neuroprotective effects of compounds with antioxidant and anti-inflammatory properties in a Drosophila model of Parkinson's disease. Bmc Neuroscience, 10.
9. KYUNG, H., KWONG, J. M., BEKERMAN, V., GU, L., YADEGARI, D., CAPRIOLI, J. & PIRI, N. 2015. Celastrol supports survival of retinal ganglion cells injured by optic nerve crush. Brain Res, 1609, 21-30.
10. ZHANG, J., ZHOU, K., ZHANG, X., ZHOU, Y., LI, Z. & SHANG, F. 2019. Celastrol Ameliorates Inflammation in Human Retinal Pigment Epithelial Cells by Suppressing NF-kappaB Signaling. J Ocul Pharmacol Ther, 35, 116-123.
11. ZHOU, Y., ZHOU, L., ZHOU, K., ZHANG, J., SHANG, F. & ZHANG, X. 2019. Celastrol Protects RPE Cells from Oxidative Stress-Induced Cell Death via Activation of Nrf2 Signaling Pathway. Curr Mol Med, 19, 172-182.
12. GUO, H. J., YANG, Y., ZHANG, Q., DENG, J. R., YANG, Y., LI, S. Q., SO, P. K., LAM, T. C., WONG, M. K. & ZHAO, Q. 2022. Integrated Mass Spectrometry Reveals Celastrol As a Novel Catechol-O-methyltransferase Inhibitor. Acs Chemical Biology.
13. CASCAO, R., FONSECA, J. E. & MOITA, L. F. 2017. Celastrol: A Spectrum of Treatment Opportunities in Chronic Diseases. Front Med (Lausanne), 4, 69.
14. HOU, W., LIU, B. & XU, H. 2020. Celastrol: Progresses in structure-modifications, structure-activity relationships, pharmacology and toxicology. Eur J Med Chem, 189, 112081.
   Myopia is a common refractive error characterized by the inability to see distant objects. It typically occurs since age 10 and then progresses (or worsens) every few months or year. It is caused by the eyes growing too fast or too long beyond their normal growth rate. However, the underlying molecular mechanism of myopia still remains unclear, and no drug has been approved by FDA for clinical use so far.

Catechol-O-methyltransferase (COMT) is a methyltransferase that metabolizes catechol estrogens and catechol neurotransmitters such as dopamine. Some COMT inhibitors have been used in combination with a precursor of dopamine, levodopa, to control motor symptoms of Parkinson's disease and other neurological disorders but their clinical application is limited by their ability to penetrate brain and adverse side effect. Extensive studies have reported that dopamine acts as a “stop” signal in ocular growth (Feldkaemper and Schaeffel, 2013a, Zhou, et al., 2017). Reduced retinal dopamine level has been found in various form-deprivation myopia (FDM) models such as chicks, rhesus monkeys, guinea pigs, and tree shrews, and also found in LIM chicks. Stone et al. (1989), Schmid and Wildsoet (2004), Dong et al. (2011), Iuvone et al. (1991) and Yan et al. (2015) have shown that a non-selective dopamine receptor agonist, apomorphine, can prevent FDM in various animal models including chickens, guinea pigs, monkeys and mice.

Celastrol, a pentacyclic triterpene with electrophilic quinone methide, is one of five promising natural products for drug discovery that can be extracted from Lei Gong Teng (root and stem of Tripterygium wilfordii, or generically called Thunder God Vine). It has been found with a broad spectrum of activities in anti-obesity, anti-cancer, anti-oxidant, anti-inflammation and neuroprotection. Its potent dopaminergic neuroprotection effect has been shown in a Drosophila Parkinson's disease model (Faust et al., 2009), but its molecular mechanism through an increased dopamine level in the brain such as binding target is not known.

Celastrol has been tested in various ocular disease models to demonstrate its potentials in treating ocular diseases, for example, in a glaucoma rat model, the average number of retinal ganglion cells (RGCs) was increased by approximately 80% and 78% with an intravitreal injection of 1 mg/kg and 5 mg/kg celastrol, compared to controls (Kyung et al., 2015). Celastrol can also inhibit inflammatory effect in human retinal pigment epithelial (RPE) cells via suppressing NF-kappaB signaling (Zhang et al., 2019). Zhou et al (2019) further demonstrated that celastrol is able to protect RPE cells from oxidative stress-induced cell death via activation of the Nrf2 signaling pathway. A recent study by Guo et al. (2022) showed that a low concentration of celastrol (1 μM) resulted in a 6-fold increase in dopamine in neuroendocrine chromaffin cells and proposed that COMT is a major binding target of celastrol identified by chemical proteomics. All these findings lead to an assumption that celastrol is able to control or modulate myopia development via up-regulating retinal dopamine expression, in turn inhibit COMT (including soluble isoform, S-COMT, and membrane-bound form, MB-COMT). However, Cascão et al. (2017) and Hou et al. (2020) showed that high dose of celastrol has adverse side effects, as they found that celastrol has limited bioavailability, undesired biodistribution, poor water stability and narrow therapeutic range.

Therefore, there is an unmet need for a clinically relevant therapeutic regime based on an application of celastrol for reducing or retarding progression of myopia.

### SUMMARY

Accordingly, in a first aspect, the present disclosure provides a method for reducing myopia progression comprising administering to a subject a compound represented by the following formula:

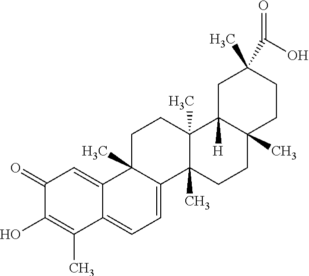

or a pharmaceutically accept salt thereof.

A second aspect of the present disclosure provides a composition comprising a therapeutically effective amount of a compound represented by the following formula:

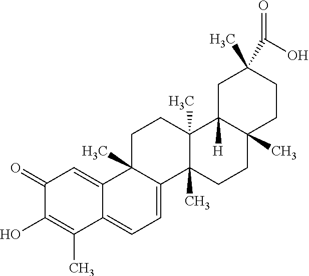

or a pharmaceutically accept salt thereof, for reducing myopia progression in a subject.

A third aspect of the present disclosure provides a method of increasing choroidal thickness of a uvea of a subject comprising contacting a compound represented by the following formula with a vitreous, cornea, or conjunctiva of the subject:

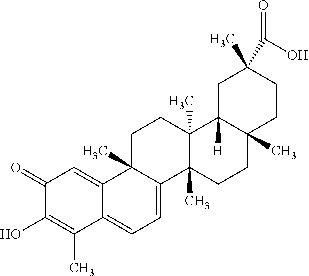

or a pharmaceutically accept salt thereof.

In certain embodiments, a therapeutically effective amount of the compound is formulated into a composition which improves bioavailability, biodistribution, and stability of the compound.

In certain embodiments, the composition is administered via intravitreal injection, intravenous injection, oral administration, or topically to the eye.

In certain embodiments, the composition further comprises a pharmaceutically acceptable carrier.

In certain embodiments, the pharmaceutically acceptable carrier is based on nanomedicines including, but not limited to, standard and core-crosslinked polymeric micelles, polymer-drug conjugate, polymer-protein conjugate, antibody-drug conjugate, dendrimeric drug, polymersome, liposome, PEGylated liposome, and organic/inorganic colloid.

In certain embodiments, the pharmaceutically acceptable carrier comprises cyclodextrins (CDs) comprising beta-cyclodextrins (β-CDs).

In certain embodiments, the subject includes non-human animals and humans.

In certain embodiments, the subject is a chicken, and the therapeutically effective amount of the compound is 74 μM which is administered once daily (q.d.) via intravitreal injection for four consecutive days.

In certain embodiments, the subject is a human, and the therapeutically effective amount of the compound is about 1.665 mM which is administered via an eyedrop once daily.

In certain embodiments, the compound contacts with the vitreous of the subject directly when the subject is a chicken.

In certain embodiments, the compound contacts with the cornea or conjunctiva of the subject via a non-invasive administrative route such as an eyedrop containing a therapeutically effective amount of the compound once daily when the subject is human.

In certain embodiments, the human subject is normally aged 18 or below.

In certain embodiments, the human subject is between 6 and 18 years old.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Other aspects of the present disclosure are disclosed as illustrated by the embodiments hereinafter.

### BRIEF DESCRIPTION OF DRAWINGS

The appended drawings, where like reference numerals refer to identical or functionally similar elements, contain figures of certain embodiments to further illustrate and clarify the above and other aspects, advantages and features of the present disclosure. It will be appreciated that these drawings depict embodiments of the disclosure and are not intended to limit its scope. The invention will be described and explained with additional specificity and detail through the use of the accompanying drawings in which:

FIG. 1 shows the scheme of an in vivo study of celastrol on a unilateral lens-induced myopia (LIM) chick model;

FIG. 2A shows the effect of celastrol on refractive error in the unilateral LIM chick model;

FIG. 2B shows the effect of celastrol on vitreous chamber depth (VCD) in the unilateral LIM chick model;

FIG. 2C shows the effect of celastrol on axial length (AL) in the unilateral LIM chick model; and

FIG. 2D shows the effect of celastrol on choroidal thickness in the unilateral LIM chick model.

Skilled artisans will appreciate that elements in the figures are illustrated for simplicity and clarity and have not necessarily been depicted to scale.

### DETAILED DESCRIPTION

### Definitions

As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In certain embodiments, organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, non-toxic ammonium, quaternary ammonium, and amine cations formed using counter ions, such as halide, hydroxide, carboxylate, sulfate, phosphate, nitrate, lower alkyl sulfonate, and aryl sulfonate. Organic bases from which salts can be derived include, for example, primary, secondary, and tertiary amines, substituted amines including naturally occurring substituted amines, cyclic amines, basic ion exchange resins, and the like, such as isopropylamine, trimethylamine, diethylamine, triethylamine, tripropylamine, and ethanolamine. In certain embodiments, the pharmaceutically acceptable base addition salt is chosen from ammonium, potassium, sodium, calcium, and magnesium salts.

As used herein, the terms “treat”, “treating”, “treatment”, and the like refer to reducing or ameliorating a disorder/disease and/or symptoms associated therewith. It will be appreciated, although not precluded, treating a disorder or condition does not require that the disorder, condition, or symptoms associated therewith be completely eliminated. In certain embodiments, treatment includes prevention of a disorder or condition, and/or symptoms associated therewith. The term “prevention” or “prevent” as used herein refers to any action that inhibits or at least delays the development of a disorder, condition, or symptoms associated therewith. Prevention can include primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

As used herein, the term “subject” refers to any animal (e.g., a mammal), including, but not limited to, humans, non-human primates, canines, felines, and rodents.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10%, ±7%, ±5%, ±3%, ±1%, or ±0% variation from the nominal value unless otherwise indicated or inferred.

It will be apparent to those skilled in the art that modifications, including additions and/or substitutions, may be made without departing from the scope and spirit of the invention. Specific details may be omitted so as not to obscure the invention; however, the disclosure is written to enable one skilled in the art to practice the teachings herein without undue experimentation.

The present disclosure provides a method for reducing myopia progression by administering a therapeutically effective amount of celastrol via intravitreal injection to a vitreous or via a non-invasive route, such as topically to the eye, e.g., as an eye drop containing a therapeutically effective amount of celastrol administered to a cornea or conjunctiva of a subject's eye. An animal model of lens-induced myopia (LIM) induced monocularly (or called unilateral LIM model) was used in the following examples for studying beneficial effects of celastrol on reducing myopia progression in terms of refractive error and ocular parameters such as vitreous chamber depth (VCD) and axial length (AL) of unilateral LIM chicks compared to the baseline (before unilateral LIM was induced). Change of body weight before and after 7-day LIM and choroidal thickness of a uvea of the celastrol group after 2-hr removal of the lens from the unilateral LIM chicks following the 7-day LIM were also quantified to show no adverse side effect but an increase in choroidal thickness induced by a low-dose celastrol. Animal dose and human equivalent dose of celastrol can also be derived from the results of the in vivo studies in the present disclosure. For instance, if celastrol is administered via the intravitreal injection, based on the difference in vitreous volume between the animal model tested and the human being, the human equivalent dose of celastrol could be determined. Taking chicken's vitreous volume as an illustrative example, if the celastrol is administered at about 74 μM via intravitreal injection to the chicken's vitreous, assuming that the vitreous volume of a chicken in general is about 200 μl, based on an estimated human vitreous volume (about 4.5 ml), the human equivalent dose of celastrol via intravitreal injection is determined to be about 1.665 mM.

In certain embodiments, celastrol is administered to the subject 1, 2, 3, 4 or more times daily for a period of 1, 2, 3, 4, 5, 6, 7, 14, 21, 28 or more days. In certain embodiments, celastrol is administered to the subject 1, 2, 3, 4 or more times daily for a period of 1-28 days, 1-21 days, 1-14 days, 1-7 days, 2-7 days, 3-7 days, 3-6 days, 2-6 days, 3-7 days, or 3-5 days.

In instances in which celastrol is formulated as an eye drop formulation and administered topically to an eye of the subject, the eye drop formulation can comprise celastrol at a concentration between 0.1-10 mM, 0.1-9 mM, 0.1-8 mM, 0.1-7 mM, 0.1-5 mM, 0.5-5 mM, 1-5 mM, 1-4 mM, 1-3 mM, 1-2 mM, 1.1-2 mM, 1.2-1.9 mM, 1.3-1.8 mM, 1.4-1.7 mM, or 1.5-1.7 mM. In certain embodiments, the eye drop formulation comprise celastrol at a concentration of about 1.665 mM. 10-200, 10-150 μl, 10-100 μl, 25-75 μl, or 25-50 μl of the eye drop formulation can be administered to an eye of the subject.

### EXAMPLES

### Example 1—In Vivo Model of Myopia and Study of the Effects of Celastrol on Myopia Progression

To create a lens-induced myopia (LIM) animal model, concave lenses of −5 D were applied to the White Leghorn chicks (Gallus gallus), hatched from specific pathogen-free eggs (SPF, Jinan, China), until they were ten days old (PN10, baseline). All the animals were raised in standard cages at 25° C. with sufficient food and water daily under the 12 h:12 h light/dark cycle. The central ambient luminance over the cages was maintained at around 260 lux.

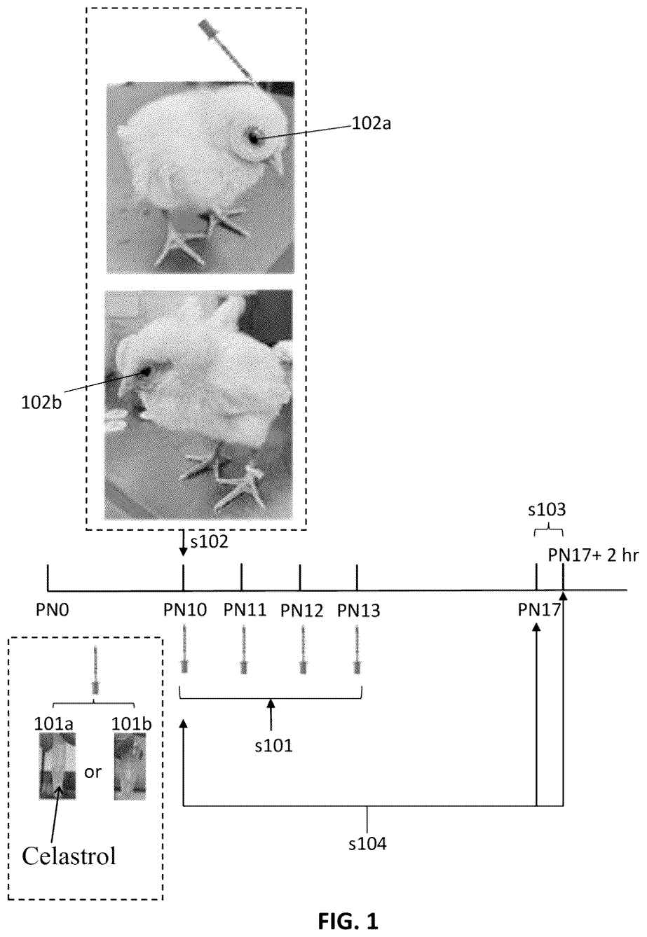

Turning to FIG. 1, starting from ten days old (PN10), the LIM chicks received four intravitreal injections in four consecutive days, i.e., PN10, PN11, PN12 and PN13 (s101). LIM chicks were randomly assigned to two groups, including LIM chicks (n=6) with vehicle injection (1% DMSO with 1% β-CDs) (101b) and LIM chicks (n=7) with celastrol injection (74 μM celastrol dissolved in 1% DMSO with 1% β-CDs) (101a), and no gender preference was selected in the experimental groupings. β-CDs were selected as a drug carrier of celastrol because of its doughnut-shaped cavities with hydrophilic outer and hydrophobic inner surfaces to form a complex with the celastrol to enhance the solubility, stability and permeation of celastrol in vivo. The freshly prepared solution (10 μl) was injected monocularly with a 0.3-mL syringe (31-gauge needle, BD Medical, Le Pont deClaix, France) into the vitreous through the sclera, choroid, and retina close to the margin of the upper orbit. Immediately following the first injection on PN10, the LIM chicks' right (or left) eyes were assigned to −5 D lenses (treated eyes 102a), and no lens was attached to the fellow eyes (control eyes 102b) (s102). The treated eyes 102a covered with lens in each LIM chick (unilateral LIM) received the subsequent celastrol injection 101a or vehicle 101b on PN11, PN12 and PN13, whereas there was no treatment for the contralateral (control) eye 102b.

After 7-day LIM (PN17), the lenses were removed for 2 hours (s103). The refractive error and ocular parameters were recorded separately at baseline (PN10), 7-day LIM (PN17), and after 2-hour recovery (PN17+2 hr) using the steak retinoscopy and high-frequency ultrasonography (s104). Refractive errors were detected using a streak retinoscope, and ocular parameters were measured using a high-frequency A-scan ultrasound system with a 30 MHz transducer (Panametrics, Inc., Waltham, MA), respectively, before and after treatments. The spherical equivalent (S.E.) was calculated from the refractive status (S.E.=spherical power+½ cylindrical power). Axial length (AL) was defined from the front of the cornea to the back of the vitreous chamber.

### Example 2—Analyses of Biometric Measurements from LIM Chicks Treated with Celastrol

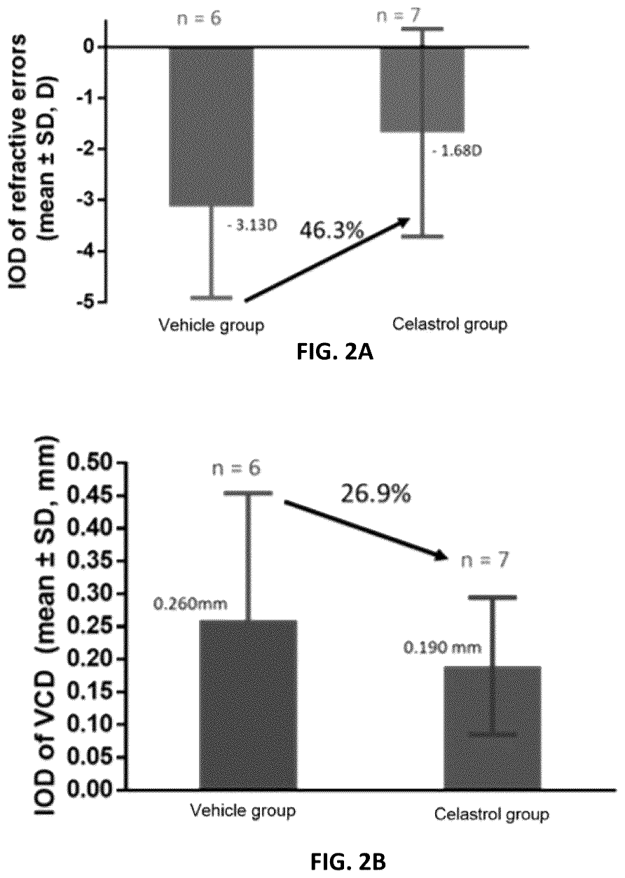

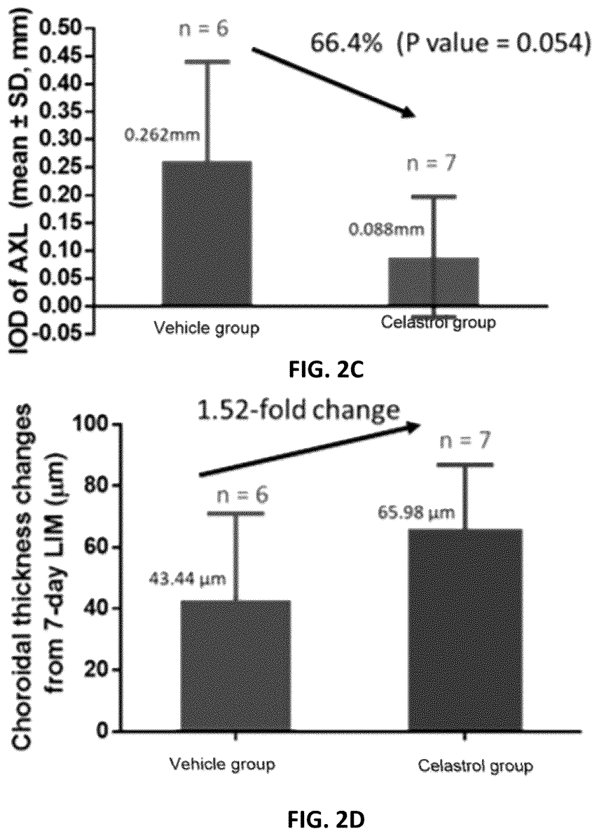

Biometric data obtained from unilateral LIM chicks treated with or without celastrol according to the scheme depicted in Example 1 and FIG. 1 were analyzed. First of all, after the 7-day lens treatment period (PN1, the interocular differences (differences between the lens-treated eyes and the contralateral fellow eyes) were calculated for the three ocular parameters (refractive error, vitreous chamber depth, and axial length), and the results are shown in FIGS. 2A-2C, respectively. Because choroid thickness change is believed to be an indicator of progression to myopia, the choroidal thickness was also measured after a 2-hour recovery, and the results are shown in FIG. 2D. The mean interocular differences and choroidal thickness changes are presented with ±SD. The significance between celastrol and vehicle groups was analyzed using an unpaired t-test with equal variance.

At the baseline (PN10), there was no significant difference in all the ocular parameters between the two groups. After 7-day LIM (PN17), the body weight of the celastrol group was similar to that of the vehicle group. There was no retardation of growth regarding body weight after celastrol treatment. For ocular biometrics, the hyperopia was decreased in both groups. An averaged shift of relative myopia (−1.45 D, mean, FIG. 2A) in interocular difference was observed in the vehicle group (celastrol vs. vehicle: −1.68±2.03 D vs. −3.13±1.78 D, mean±SD). Compared to the celastrol group, A-scan also confirmed the corresponding enlargement of the vitreous chamber depth (celastrol group vs. vehicle group: 0.190±0.105 mm vs. 0.260±0.194 mm, mean±SD) and elongation of axial length (AL) (celastrol vs. vehicle: 0.088±0.109 mm vs. 0.262±0.178 mm, mean±SD, P=0.054) in the vehicle group. A numerical elongation of AL (0.174 mm, mean) and enlargement of VCD (0.070 mm) were also observed in the vehicle group compared with the celastrol group, although the difference did not reach statistical significance (P>0.05, FIGS. 2B and 2C). Notably, after 2-hour recovery from 7-day LIM (PN17+2 hr), choroidal thickness was increased by about 1.52-fold in the celastrol group compared with the vehicle group (celastrol group vs. vehicle group: 65.98±20.89 μm vs. 43.44±27.50 μm, mean±SD, P=0.072, FIG. 2D). Based on refractive error and axial length data, approximately 46.3% and 66.4% inhibition occurred in the celastrol group. Celastrol could suppress myopia progression through intravitreal injection at the dose tested.

From the results in this example, it is suggested that celastrol could suppress myopia progression at the low dose tested (74 μM in unilateral LIM chick model). Based on refractive error and axial length data, about 43% and 46.2% inhibition occurred in the celastrol group compared to the vehicle group, which is comparable to atropine treatment. It is also suggested that celastrol can induce choroidal thickness, i.e., an increase in choroidal thickness by approximately 1.5 times after a two-hour recovery from myopia, which is an indication of protective effect against myopia, in turn, inhibit myopia progression. Celastrol may also serve as an anti-oxidant, exert anti-inflammation and neuroprotective effects on the subject through up-regulating expression of dopamine in a particular region or tissue.

Although the invention has been described in terms of certain embodiments, other embodiments apparent to those of ordinary skill in the art are also within the scope of this invention. Accordingly, the scope of the invention is intended to be defined only by the claims which follow.

What is claimed is:

1. A method for reducing myopia progression comprising administering to a subject a composition comprising a therapeutically effective amount of a compound represented by the following formula: 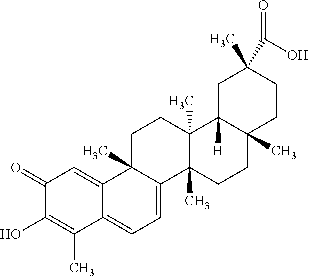
or a pharmaceutically accept salt thereof.

2. The method of claim 1, wherein the composition is administered via intravitreal injection, intravenous injection, oral administration, or topically to the eye.

3. The method of claim 1, wherein the composition further comprises a pharmaceutically acceptable carrier.

4. The method of claim 1, wherein the subject comprises a non-human animal or a human.

5. The method of claim 1, wherein the subject is a chicken, and the therapeutically effective amount of the compound is 74 μM which is administered once daily via intravitreal injection for four consecutive days.

6. The method of claim 1, wherein the subject is a human, and the therapeutically effective amount of the compound is 1.665 mM which is administered via an eyedrop once daily.

7. A composition comprising a therapeutically effective amount of a compound represented by the following formula: 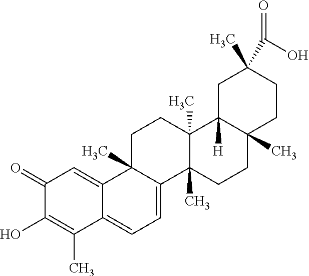
or a pharmaceutically accept salt thereof, for reducing myopia progression in a subject.

8. The composition of claim 7, being administered via intravitreal injection, intravenous injection, oral administration, or topically to the eye.

9. The composition of claim 7, further comprising a pharmaceutically acceptable carrier.

10. The composition of claim 7, wherein the subject comprises non-human animals and humans.

11. The composition of claim 7, wherein the subject is a chicken, and the therapeutically effective amount of the compound is 74 μM which is administered once daily via intravitreal injection for four consecutive days.

12. The composition of claim 7, wherein the subject is a human, and the therapeutically effective amount of the compound is 1.665 mM which is administered via an eyedrop once daily.

13. A method of increasing choroidal thickness of a uvea of a subject comprising contacting a compound represented by the following formula with a vitreous, cornea, or conjunctiva of the subject: 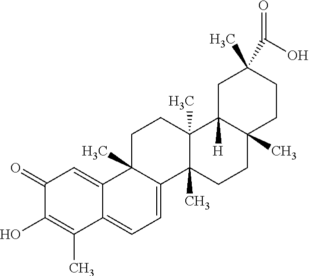
or a pharmaceutically accept salt thereof.

14. The method of claim 13, wherein the compound is formulated into an injectable form or as an eyedrop.

15. The method of claim 13, wherein the subject comprises non-human animals and humans.

16. The method of claim 13, wherein the subject is a chicken, and the compound contacts with the vitreous of the subject at 74 μM once daily for four consecutive days.

17. The method of claim 13, wherein the subject is a human, and the compound contacts with the cornea or conjunctiva of the subject via an eyedrop containing a therapeutically effective amount of the compound once daily.
